(19) United States
(12) Patent Application Publication
(10) Pub. No.: US 2020/0176086 A1
(43) Pub. Date: Jun. 4, 2020

(54) Method for Compiling A Genomic Database for A Complex Disease And Method for Using The Compiled Database to Identify Genetic Patterns in The Complex Disease to Establish Diagnostic Biomarkers

(71) Applicant: Nova Southeastern University, Fort Lauderdale, FL (US)

(72) Inventors: Nancy KLIMAS, Fort Lauderdale, FL (US); Kelly HILTON, Fort Lauderdale, FL (US); Kristina GEMAYEL, Fort Lauderdale, FL (US)

(73) Assignee: Nova Southeastern University, Fort Lauderdale, FL (US)

(21) Appl. No.: 16/624,320

(22) PCT Filed: Jun. 29, 2018
(86) PCT No.: PCT/US2018/040418

§ 371 Date: Dec. 19, 2019

Related U.S. Application Data
(60) Provisional application No. 62/526,441, filed on Jun. 29, 2017.

(51) Int. Cl. G16B 50/30; G16H 20/10; G16B 20/20; G16H 10/20

(52) U.S. Cl. CPC G16B 50/30; G16H 10/20; G16B 20/20; G16H 20/10

(57) ABSTRACT

The invention provides a method for compiling a genomic database for a complex disease, such as Myalgic Encephalomyelitis/Chronic Fatigue Syndrome (ME/CFS), and a method for using the database for identifying genetic patterns that can potentially categorize, diagnose, and/or predict therapeutics for the complex disease.

### FIELD OF THE INVENTION

The invention generally relates to medical genomics, particularly to the use of genomics to characterize, diagnose, and/or treat complex diseases, and most particularly to a genetic database for Myalgic Encephalomyelitis/Chronic Fatigue Syndrome (ME/CFS) and methods for using the database for identification of genetic patterns for potential diagnostics of ME/CFS.

### BACKGROUND

Myalgic Encephalomyelitis/Chronic Fatigue Syndrome (ME/CFS) is a complex disease with unknown causes and is characterized by severe debilitating fatigue that is accompanied by symptoms of impaired concentration, impaired short-term memory, disturbed sleep patterns, post-exertional malaise, and flu-like symptoms. A significant portion of patients exhibit immune dysfunction (Maher et al. Bull IACFS ME. 2008 FALL; 16(3):19-33). Studies suggest that quality of life is particularly and uniquely disrupted in CFS (Evengard et al. Drugs. 2002; 62(17):2433-2446). Patients report a high degree of disability and a moderate degree of emotional or psychological ill-health. Approximately 24% of patients are housebound and unable to work. Thus, ME/CFS creates both personal and economic loss. Spontaneous recovery is relatively rare in patients not responding to treatment (Van der Wed J Psychosom Res. 2002 September; 53(3):749-753).

While there have been references to a similar illness in the medical literature over several centuries, recent research efforts are based on the study of a population defined by the consensus CFS case definition, which was first published in 1988 (Holmes 1991).

In 1994 an international collaborative group, which included the authors of the previous case definitions, published the current International CFS Research Case Definition (Fukada et al. 1994) also by consensus. This definition recommended careful use of exclusion criteria, and stratified according to so-called “essential subgrouping variables” i.e. co-morbid medical or neuropsychiatric conditions, fatigue severity, fatigue duration, and functional status.

More recently, investigators have attempted to define CFS subgroups based on their reported symptoms using statistical techniques such as latent class analysis, factor analysis, and cluster analysis. The study by Hickie et al (1995) identified two patient groups: one reporting symptoms of somatoform disorders and the other reporting neuropsychological symptoms. These two patient groups were clinically heterogeneous and the authors suggested different etiologies for each group.

A cluster analysis of symptoms from a community-based study in Chicago (Jason et al. 2002) revealed two clusters of distinct CFS patients: one characterized by most severe post-exertional fatigue and most improvement in fatigue following rest. Another population-based study, in Witchita, also identified two clusters of CFS patients that discriminated between the well and most unwell individuals (Nisenbaum, 2003). However, symptoms might not be sufficient to fully discriminate CFS subgroups. Two reviews concluded that there was substantial overlap between CFS symptoms and symptoms of other unexplained chronic illness (Wessley, 1999; Aaron, 2001).

It is clear that researchers have found the 1994 criteria difficult to use and to apply uniformly, and no particular criteria for subgrouping has been uniformly accepted by investigators. There is a concern among investigators that interpretive differences have let to studies that are not really comparable. Therefore, considering the above studies, it is clear that other variables need to be considered to make the CFS classification more specific.

Most of the research in the ME/CFS field has not previously been aimed at genetic studies, but recently there has been a push towards this direction. The studies involving genes up to this point have centered around two specific and common symptoms in patients afflicted with ME/CFS; pain and fatigue enhancement, sometimes referred to as post-exertional malaise. The two pioneers in this field are currently Dr. Alan Light and Dr. Dane Cook. Dr. Alan Light's lab focuses on the neurobiology in pain pathways and has characterized the role of acid-sensing ion channels in muscle fatigue and pain (Light et al. J. Pain 10:1099-1112). Dr. Dane Cook is utilizing gene expression studies to determine the etiology of post-exertional malaise, while comparing the genetic expression to brain-imaging techniques and bloodwork before and after administrating an exercise challenge (Meyer et al. Post-Exertion Malaise in Chronic Fatigue Syndrome: Symptoms and Gene Expression. Fatigue: Biomedicine, Health, & Behavior. 1(4):190-209 2013).

It is known that single nucleotide polymorphisms (SNPs) play an important role in gene expression changes that can manifest as phenotypic changes. Most SNPs have no effect on health or development. However, some of the genetic differences resulting from SNPs have found to be associated with complex diseases such as ME/CFS. Using novel methods such as bioinformatics analysis, and genome testing, SNPs can be quickly determined in a large sample of participants. The disparity in genomic research in the ME/CFS field is apparent, and SNP identification can offer a potential pathway to valuable insight on the disease process and symptomology in these patients.

### SUMMARY OF THE INVENTION

The research leading to this invention is a prospective multi-site Myalgic Encephalomyelitis/Chronic Fatigue Syndrome (ME/CFS) study with the purpose to develop a de-identified subject population genetic database using publicly-available genetic testing sites, linked to a clinical database, to utilize for future research discovery. Analysis strategies using this database will help investigators to develop subgroup criteria for future genetic discoveries linked to ME/CFS.

Objectives of this study are multifaceted and intend to systematically apply a set of instruments to assess the domains of ME/CFS and related syndromes, including severity of illness, function, co-morbid, and exclusionary conditions. Assessment tools will be implemented using a computer/web-based format, de-identified genetic data will be collected from the ME/CFS population through the utilization of social media, and a database for future work in this area will be maintained.

Thus, the instant invention provides a database, prepared by a patient-scientist partnership, that combines genetic data from Myalgic Encephalomyelitis/Chronic Fatigue Syndrome (ME/CFS) patients which will show whether there is a genetic pattern that would diagnose ME/CFS and/or define biologic subsets; determine genetic risk to develop ME/CFS; and/or better explain the causes of and predict therapies for ME/CFS. Why would one person recover from a common infection and the next spin into a chronic, disabling illness? Does the genetic signature provide clues to predict therapies? It is hoped that genetic analysis, using the inventive database, could provide answers. Furthermore, creation of the database may lead to both development of a standard diagnostic test, such that the time and cost of establishing a diagnosis is reduced, and to effective, tailored treatments, such that housebound disability is reduced to enhance patient quality of life.

One genetic pattern useful in analysis in the collection of participant data is single nucleotide polymorphisms (SNPs). SNPs are the most common type of genetic variation (from the norm) among people. Each SNP represents a difference in a single DNA building block, i.e. a single nucleotide. For example, a SNP may replace the normal nucleotide cytosine (C) with the nucleotide thymine (T) in a certain stretch of DNA. SNPs occur normally throughout a person's DNA. They occur about once in every 300 nucleotides on average, which means there are approximately 10 million SNPs in the human genome. Most commonly, these variations are found in the DNA between genes. They can act as biological markers, helping scientists locate genes that are associated with disease. It is known that SNPs play an important role in gene expression and can manifest in phenotypic changes. Most SNPs have no effect on health or development. Some of these genetic differences, however, have been proven to be very important in the study of human health. When SNPs occur within a gene or in a regulatory region near a gene, they may play a more direct role in disease by affecting the gene's function. SNPs may help predict: an individual's response to certain drugs, an individual's susceptibility to environmental factors such as toxins, and/or an individual's risk of developing particular diseases. SNPs can also be used to track the inheritance of disease genes within families.

The study described herein will work to identify SNPs associated with complex diseases such as ME/CFS and determine if the SNPs point to potential diagnostic tests, potential treatments, and/or subgrouping strategies.

In a general aspect, the invention relates to compilation of data into databases. In this regard, the disclosed online recruitment methods can be used in a wide variety of research studies. The streamlining of the recruitment steps can be tailored to the needs of the research investigator.

In another general aspect, the invention relates to methods for improving diagnosis and treatment of complex diseases. A “complex disease” does not result from a single factor, but rather from multiple factors and often manifests from an interaction of genetic, environmental, and lifestyle factors. Thus, although complex diseases appear to run in families, they are not attributable to genetics alone. Because of this complex etiology, complex diseases are usually difficult to diagnose and treat. Non-limiting examples of complex diseases are Myalgic Encephalomyelitis/Chronic Fatigue Syndrome (ME/CFS), cancer, obesity, and schizophrenia.

In yet another general aspect, the invention includes utilizing social media as a platform to reach a large sample size of study participants to alleviate the costs and burdens of research study recruitment.

In one aspect, the invention provides a method for preparing a genomic database for a complex disease, such as but not limited to, Myalgic Encephalomyelitis/Chronic Fatigue Syndrome (ME/CFS). The method includes steps of selecting a complex disease; recruiting patients diagnosed with the selected complex disease using online platforms; requesting donation of genomic data from the recruited patients; collecting and de-identifying donated genomic data from the recruited patients; and storing collected genomic data on a computer-readable medium, thereby preparing the genomic database for the complex disease. Additionally, the method can include collection of data from the patients through use of an on-line questionnaire. For example, a standardized CFS questionnaire via a secure online program such as REDCap (Research Electronic Data Capture) is used in the study described herein. Utilization of a questionnaire with the method includes steps of using at least one computer to prepare a questionnaire including questions about the selected complex disease, such as, but not limited to questions about symptoms, disease severity, disease onset, mental health, sleep patterns, social history, and environment; requesting completion of the questionnaire by the recruited patients; collecting completed questionnaires from the recruited patients; and storing the completed questionnaires on a computer-readable medium.

In another aspect, the invention encompasses genomic databases prepared by the methods described herein, such as, but not limited to, a genomic database for Myalgic Encephalomyelitis/Chronic Fatigue Syndrome (ME/CFS).

In yet another aspect, the invention includes a method for identifying relevant frequencies of single nucleotide polymorphisms (SNP) in a disease population. The method includes steps of preparing a genomic database according to the methods described herein; identifying complex disease-specific single nucleotide polymorphisms (SNPs) in the genomic database; calculating the relevant frequencies of the identified complex disease-specific SNPs; and comparing frequencies of the identified complex disease-specific SNPs to a healthy population.

In another similar aspect, the invention includes a method for identifying relevant frequencies of single nucleotide polymorphisms (SNPs) associated with a complex disease in a disease population. The method includes steps of preparing a genomic database according to the methods described herein; identifying single nucleotide polymorphisms (SNPs) in the genomic database; calculating the relevant frequencies of the identified SNPs; and comparing frequencies of the identified SNPs to a healthy population to determine association with the complex disease.

A non-limiting example of SNPs to be identified by these methods are those located on chromosome 11 of the methylenetetrahydrofolate reductase (MTHFR) gene in patients having Myalgic Encephalomyelitis/Chronic Fatigue Syndrome (ME/CFS). SNPs can be correlated with patient symptoms disclosed on the questionnaires and/or a pattern of multiple SNPs can provide disease diagnosis. ME/CFS could be the result of a large number of SNP variants working together.

In still another aspect, the invention includes a method for determining susceptibility to Myalgic Encephalomyelitis/Chronic Fatigue Syndrome (ME/CFS) in a subject. The subject is generally a human being but is not limited thereto. A “subject” can also be referred to as a patient, as an individual, and/or as a participant. The method includes steps for determining genotype of the subject at chromosome 11 and identifying a single nucleotide polymorphism (SNP) at any of positions chromosome 11, 800, 251; chromosome 11, 801, 166; chromosome 11, 794, 766; and chromosome 11, 795, 161. The identification of an SNP at any of these locations indicates susceptibility to ME/CFS in the subject.

A different aspect of the invention is a method for prophylactically treating a subject for Myalgic Encephalomyelitis/Chronic Fatigue Syndrome (ME/CFS). The method involves determining susceptibility to Myalgic Encephalomyelitis/Chronic Fatigue Syndrome (ME/CFS) in the subject; and if the identification of a SNP indicates susceptibility to ME/CFS in the subject, then treating the subject for Myalgic Encephalomyelitis/Chronic Fatigue Syndrome (ME/CFS). Treating the subject includes at least one of establishing a daily exercise routine, managing diet and nutrition, administering vitamins and supplements, and administering prescription medication.

In another aspect, the invention provides a bioinformatics analysis of SNPs in the MTHFR gene and the folate methylation pathway.

Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

In this regard, it should be emphasized that although ME/CFS is used throughout the specification as a non-limiting example, one of ordinary skill in the art would understand that the disclosure can be applied to other complex diseases including, but not limited to heart disease, diabetes, Alzheimer disease, autism, Parkinson disease, asthma, and spina bifida.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIGS. 1A-B are pie charts illustrating data collected from the Karnofsky Performance Scale portion of the ME/CFS questionnaire.

FIGS. 2A-D are pie charts illustrating data collected from the CFS symptoms portion of the ME/CFS questionnaire.

FIGS. 3A-C are graphs quantifying the number of patients identifying with particular symptoms noted in the CFS symptoms portion of the questionnaire.

FIG. 4 illustrates an SNP identified in a portion of DNA (SEQ ID NOS:1-3).

FIG. 5 shows data revealing SNP Frequencies Identified (SEQ ID NO:4).

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described databases, methods, techniques, diagnostics, and any further application of the principles of the invention as described herein, are contemplated as would normally occur to one skilled in the art to which the invention relates.

In order to effectively move the field of CFS diagnostics and treatment forward, studies must be designed to eliminate previous limitations and setbacks. Such studies should include investigation into various genomic mutation patterns with large (in the 1000s) enough samples to address these issues. To circumvent the limitations of previous studies, the instant inventors decided to recruit patients beyond the clinicians' private practice, and even beyond regional geographic locations. The broader the geographic region, the more meaningful are the results obtained. Additionally, such recruitment techniques can open the study to racial or socioeconomic demographic biases. Therefore, since CFS has shown no particular geographic preference, this study also has no geographic preference. The instant inventors have decided to recruit globally through the use of social media with the intention of having a racially diverse subject population and a large sample size. A recruitment video, explaining the goals and scope of this project, is posted on frequently visited ME/CFS websites. In addition, the project has a Facebook page and the instant inventors plan to have a press release at Phoenix Rising, a respected ME/CFS informative website. 400+ participants (with participants from the USA, Australia, New Zealand, England, Italy, and Ireland) enrolled in the study purely through word of mouth and Facebook. 1000+ participants are expected to enroll after release of the recruitment video, press release, and participant recruitment poster.

The success of the project relies on the generosity of the participants donating their raw genetic data. The cost of genetic testing can range from the 100s to 1000s of dollars per person. Thus, for the number of participants expected, the cost of the project will exceed the NIH funding cap. A project of this magnitude is made possible by the very active and supportive ME/CFS patient population that is generously willing to donate their genetic information in hopes to discover answers to their condition. Therefore, crowdsourcing efforts place a pivotal role in this project.

Detailed characterization of the CFS population should be conducted to permit clinical subgrouping. In an embodiment, it is intended to subgroup the participants enrolled in this study through the utilization of a standardized CFS questionnaire via a secure online program, REDCap (Research Electronic Data Capture). The raw genomic data is collected in a standardized, de-identified manner, to ensure confidentiality for the participants. Access to the genomic data, in addition to the symptom questionnaire, will allow selection of subjects for analysis of single nucleotide polymorphisms (SNPs), for example, SNPs within the folate methylation pathway, specifically the methylenetetrahydrofolate reductase (MTHFR) gene. Patients clinically diagnosed with ME/CFS based on the Canadian Consensus Criteria for CFS have been shown to carry statistically significant pathogenic mutations in the MTHFR gene, on chromosome 11. The NCBI Genomic Database will be used in a streamline manner with genomic software called DNANexus to correctly identify pathogenic SNPs in the MTHFR gene.

One goal of this study is to identify a ME/CFS biomarker in the folate methylation pathway. Since this study involves crowdsourcing, it is limited by the lack of a confirmatory diagnostic test by a professional to determine if an individual does, in fact, have ME/CFS. To circumvent this issue, and to help validate results, the genomic data, as a whole, will be initially analyzed versus a healthy control. After analysis, the data will be separated to determine if there is any difference in genomic alterations between those patients who are professionally-diagnosed versus self-diagnosed.

To begin the project, the instant inventors created an online REDcap Platform with an appropriate ME/CFS questionnaire and obtained IRB (Institutional Review Board) approval for this first phase of the study and for recruitment of healthy control participants. The ME/CFS questionnaire starts with a series of questions for determining a potential participant's eligibility in the study, then if determined eligible, the participant/patient provides informed consent for study participation, uploads raw genetic data into the database, and answers questions divided into a series of categories including: fatigue history; CFS symptoms; multidimensional fatigue inventory; Karnofsky Performance Scale; pain inventory; questions concerning general health; HAD Scale; sleep assessment questionnaire; and questions concerning social and environmental history.

Additionally, the inventors are pursuing a partnership with 23andMe to be granted access to their healthy population database to expedite the recruitment process. Analysis of healthy controls will follow the same protocol as the diseased population, which will give better insight into the data gathered and enable clear conclusions. At the time of release of the questionnaire, the study included 400+ participants. This patient population is predominantly female, with 84.5% of participants identifying with this gender. The dominant majority, 99.4%, of participants categorize themselves in the racial group “white.” 97.3% of the participants stated they were diagnosed by a physician or health care provider as having CFS with 83.4% providing documentation of the diagnosis.

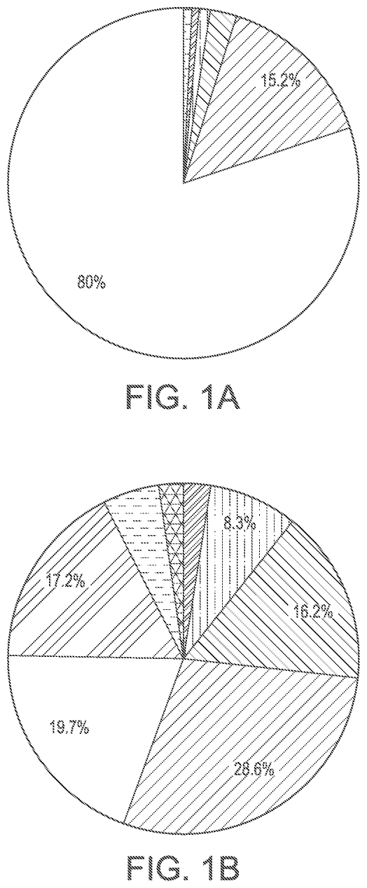

FIGS. 1A-B are pie charts illustrating data collected from the Karnofsky Performance Scale portion of the ME/CFS questionnaire. The Karnofsky Performance scale attempts to quantify fatigue. FIG. 1A shows that 95.2% of patients disagree or strongly disagree with the statement “physically being able to take on a lot.” FIG. 1B shows that 28.6% of patients identified with the activity description “your energy only allows you to do about 3 tasks per day (2-3 hours of activity). Your energy is easily drained. Thought processes are difficult. Your exercise tolerance is poor, i.e. walking up stairs is difficult.” FIG. 1B also shows that 19.7% of patients identified with the activity description “you can only perform 2 light tasks per day. Physical exercise is not tolerable. Your thought processes are very slow, and your memory is poor.”

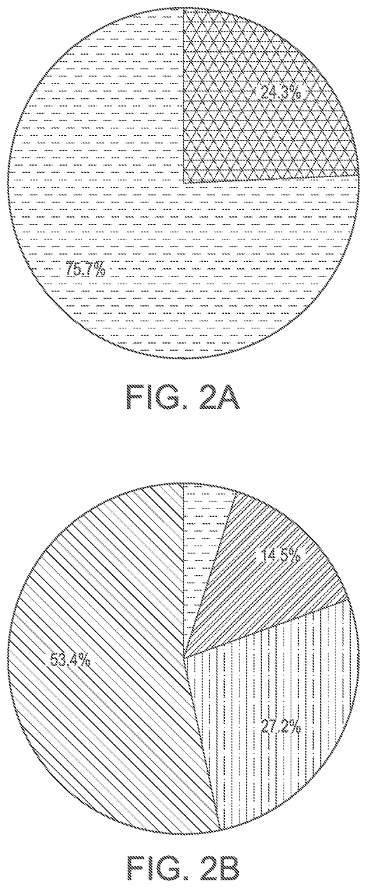

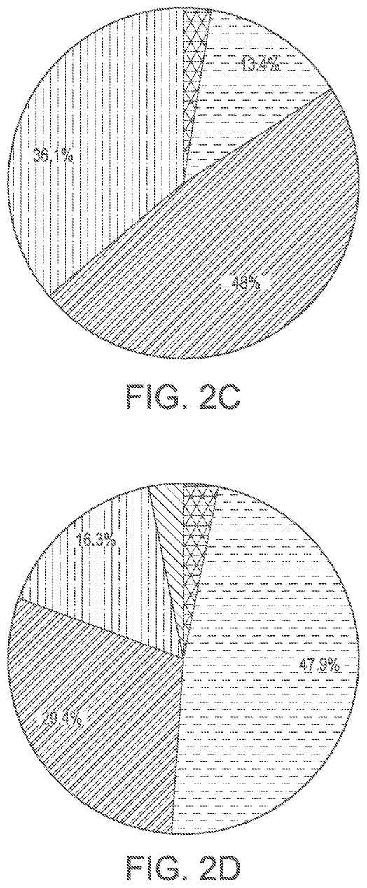

FIGS. 2A-D are pie charts illustrating data collected from the CFS symptoms portion of the ME/CFS questionnaire. FIG. 2A shows that 75.7% of patients deny sleeping all day and staying up all night. FIG. 2B shows that 80.6% of patients strongly or mostly deny feeling so down in the dumps that nothing could cheer them up. FIG. 2C shows that 84.1% of patients feel cheerful most of the time. FIG. 2D shows that 77.3% of patients consider themselves “a happy person.”

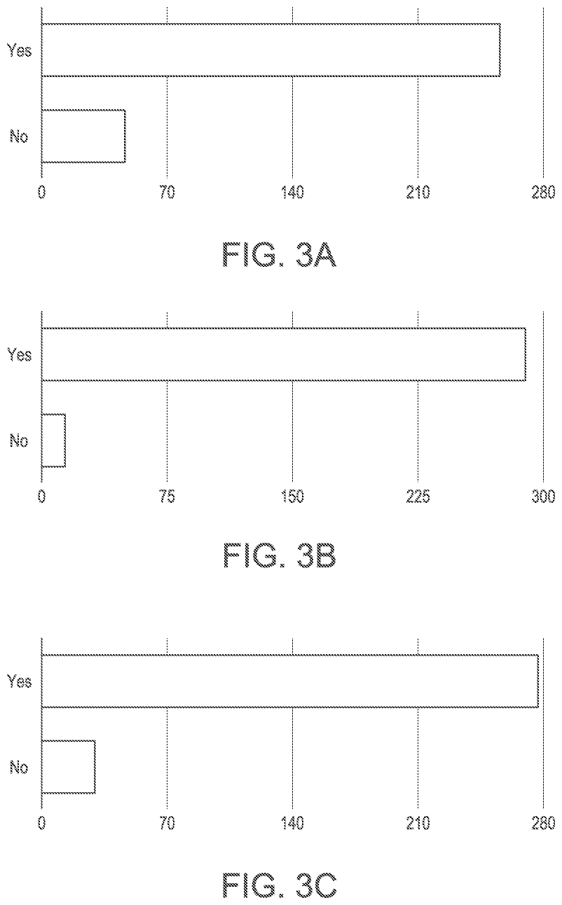

FIGS. 3A-C are graphs quantifying the number of patients identifying with particular symptoms noted in the CFS symptoms portion of the questionnaire. FIG. 3A shows that a strong majority of patients reported being absent minded or having forgetfulness. FIG. 3B shows that a strong majority of patients reported having problems remembering things. FIG. 3C shows that a strong majority of patients reported having difficulty paying attention.

After obtaining the participants' responses and genomic data, an IRB for the second phase the project will be submitted. The second phase includes bioinformatics analysis of SNPs in the MTHFR gene and the folate methylation pathway. The statistically significant pathogenic mutations found within the ME/CFS participants' genome will be compared to gene expression studies that have been done at the Institute for NeuroImmune Medicine at Nova Southeastern University (Fort Lauderdale, Fla. US). In order to establish an SNP as a biomarker for the disease process that ensues in ME/CFS patients, it must be determined that the pathogenic mutations seen in the MTHFR gene are predictive of an abnormal gene product leading to a downregulated pathway.

Using the raw genetic data of participants who meet the clinical criteria of the Canadian Consensus Chronic Fatigue Syndrome Criteria, the SNPs will then be compared for pathogenic mutations at locations: Chromosome 11, 800, 251; Chromosome 11, 801, 166; Chromosome 11, 794, 766; and Chromosome 11, 795, 161. These four locations were selected after a review of the literature for the SNPs documented to be located in the MTHFR gene. These chromosomal locations are critical for normal function of the MTHFR gene, and an SNP in these locations will result in a non-functional transcript and gene product, increasing susceptibility to various disease processes. After genetic analysis, the hypothesis based on the prevalence of statistically significant pathogenic mutations within the ME/CFS participant population will be accepted or rejected.

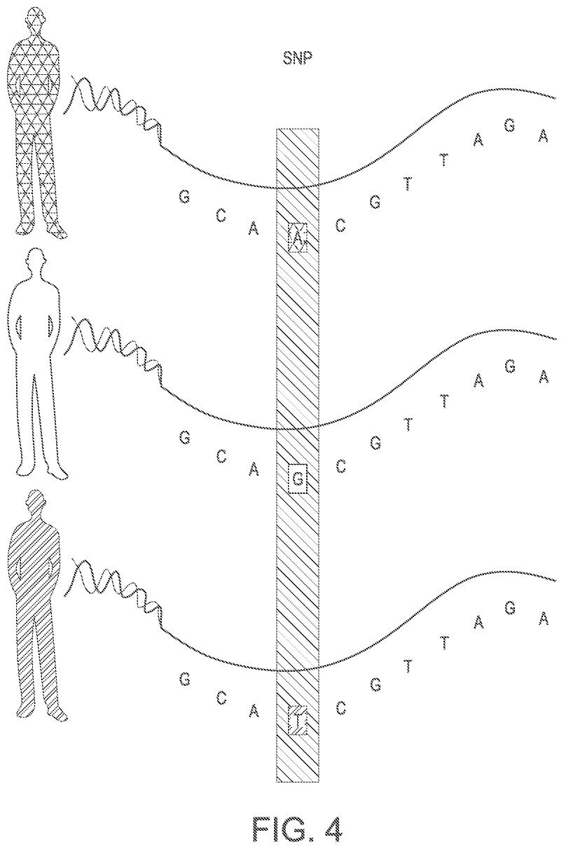

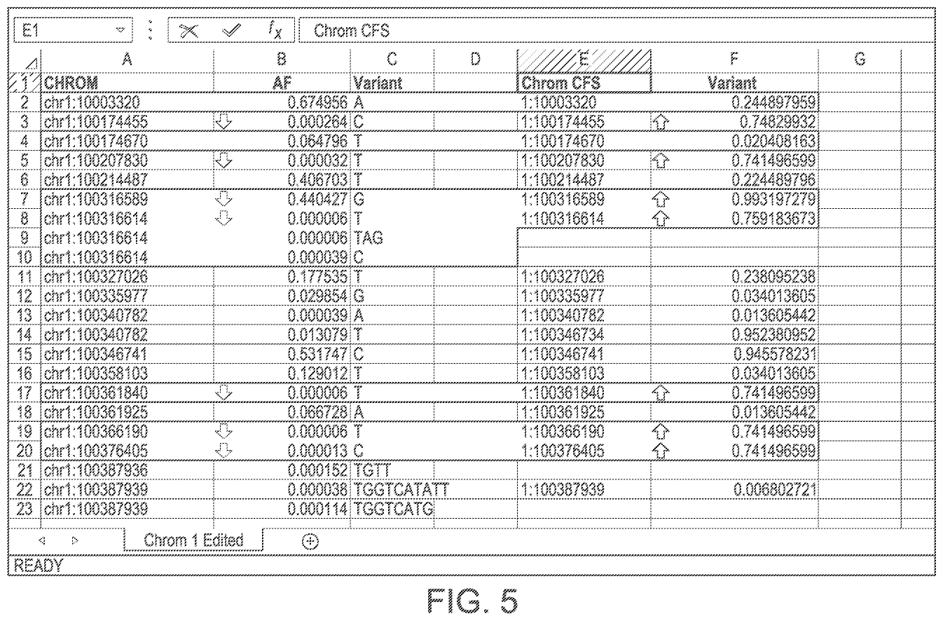

The uploaded de-identified genetic data entries acquired from REDCap are modified to a suitable format for Seattle Sequence Annotation 138. The annotated data is then filtered to include only non-synonymous and nonsense SNPs from protein coding regions (exons), microRNAs, and SNPs that are close to splice sites. FIG. 4 illustrates an SNP identified in a portion of DNA (SEQ ID NOS:1-3). The frequency of each SNP (FIG. 5) is then calculated with our cohort, based on a matrix of 1 and 0s. The frequencies of our cohort have been compared with frequencies in public databases.

Ongoing recruitment for submission of de-identified genetic data leads to a constantly increasing sample size for continual application of the aforementioned methods. This continual application will lead to identification of new SNPs in the patients as well as potentially increasing the frequency of certain SNPs. Additional investigation of a larger sample size will allow for validation of SNP trend significance found in the participant population relative to existing SNP data acquired from public databases of the general public.

Accomplishment of the genetic database described in this specification will facilitate future genetic studies and collaborative efforts geared towards improving quality of life and future outcomes for ME/CFS patients.

The disclosure includes prophylactically treating a subject for Myalgic Encephalomyelitis/Chronic Fatigue Syndrome (ME/CFS). Once the subject has been identified as being susceptible to Myalgic Encephalomyelitis/Chronic Fatigue Syndrome (ME/CFS) by the identification of SNP or a particular combination of SNPs in the subject, the subject is treated for Myalgic Encephalomyelitis/Chronic Fatigue Syndrome (ME/CFS) using any known treatment or combination of treatments. These treatments include, but are not limited to establishing an exercise regime, controlling diet and nutrition, administering vitamins and supplements, and administering prescription medications.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The methods, genomic databases, diagnostics, procedures, and techniques described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

What is claimed is:

1. A method for preparing a genomic database for a complex disease, the method comprising:
selecting a complex disease;
recruiting patients diagnosed with the selected complex disease using online platforms;
requesting donation of genomic data from the recruited patients;
collecting and de-identifying donated genomic data from the recruited patients; and
storing collected genomic data on a computer-readable medium, thereby preparing the genomic database for the complex disease.

2. The method of claim 1, wherein the complex disease is Myalgic Encephalomyelitis/Chronic Fatigue Syndrome (ME/CFS).

3. The method of claim 1, further comprising:
using at least one computer to prepare a questionnaire including questions about the selected complex disease;
requesting completion of the questionnaire by the recruited patients;
collecting completed questionnaires from the recruited patients; and
storing the completed questionnaires on a computer-readable medium.

4. The method of claim 3, wherein the questionnaire includes questions regarding disease symptoms and disease severity.

5. The method of claim 4, wherein the complex disease is Myalgic Encephalomyelitis/Chronic Fatigue Syndrome (ME/CFS).

6. A genomic database for a complex disease prepared according to the method of claim 1.

7. A genomic database for a complex disease prepared according to the method of claim 4.

8. A method for identifying relevant frequencies of single nucleotide polymorphisms (SNPs) associated with a complex disease in a disease population, the method comprising:
preparing a genomic database according to claim 1;
identifying single nucleotide polymorphisms (SNPs) in the genomic database;
calculating the relevant frequencies of the identified SNPs; and
comparing frequencies of the identified SNPs to a healthy population to determine association with the complex disease.

9. The method of claim 8, wherein the complex disease is Myalgic Encephalomyelitis/Chronic Fatigue Syndrome (ME/CFS).

10. The method of claim 9, wherein the identified single nucleotide polymorphisms (SNPs) are located on chromosome 11 of the methylenetetrahydrofolate reductase (MTHFR) gene.

11. A method for determining susceptibility to Myalgic Encephalomyelitis/Chronic Fatigue Syndrome (ME/CFS) in a subject, the method comprising:
determining genotype of the subject at chromosome 11; and
identifying a single nucleotide polymorphism (SNP) at any of positions chromosome 11, 800, 251; chromosome 11, 801, 166; chromosome 11, 794, 766; and chromosome 11, 795, 161,
wherein identification of a SNP indicates susceptibility to ME/CFS in the subject.

12. A method for prophylactically treating a subject for Myalgic Encephalomyelitis/Chronic Fatigue Syndrome (ME/CFS), the method comprising:
determining susceptibility to Myalgic Encephalomyelitis/Chronic Fatigue Syndrome (ME/CFS) in the subject with the method of claim 11; and
if the identification of a SNP indicates susceptibility to ME/CFS in the subject, then treating the subject for Myalgic Encephalomyelitis/Chronic Fatigue Syndrome (ME/CFS).

13. The method of claim 12, wherein treating the subject includes at least one of establishing a daily exercise routine, managing diet and nutrition, administering vitamins and supplements, and administering prescription medication.

14. The method of claim 13, wherein treating includes administering prescription medication.


SEQUENCE LISTING (4 sequences)

SEQ ID NO: 1 - DNA, 11 bp, Homo sapiens
gcaacgttaga

SEQ ID NO: 2 - DNA, 11 bp, Homo sapiens
gcagcgttaga

SEQ ID NO: 3 - DNA, 11 bp, Homo sapiens
gcatcgttaga

SEQ ID NO: 4 - DNA, 10 bp, Homo sapiens
tggtcatatt